(19) United States
(12) Patent Application Publication
(10) Pub. No.: US 2016/0193377 A1
(43) Pub. Date: Jul. 7, 2016

(54) Dispensing Device with Wick Retainer

(71) Applicant: beautyAvenues LLC, Reynoldsburg, OH (US)

(72) Inventors: Chad E. Lagace, Cohoes, NY (US); Matthew Chateauvert, Larchmont, NY (US); Heather Mastny, Dublin, OH (US)

(21) Appl. No.: 15/068,897

(22) Filed: Mar. 14, 2016

Related U.S. Application Data
(63) Continuation of application No. 14/334,042, filed on Jul. 17, 2014.

(51) Int. Cl. A61L 9/12

(52) U.S. Cl. CPC A61L 9/127

(57) ABSTRACT

A dispensing device dispenses volatile material into the atmosphere. The dispensing device has a reservoir for holding the volatile material, and a wick in contact with the volatile material and the atmosphere. A wick retainer is positioned in an opening of the reservoir. The wick retainer has one or more pivoting engagement members mounted in a retainer body. The engagement members pivot into engagement with the wick and retain it within the reservoir.

### CROSS REFERENCE TO RELATED APPLICATION

This application is a continuation of and claims priority to U.S. patent application Ser. No. 14/334,042, filed Jul. 17, 2014 and hereby incorporated by reference herein.

### FIELD OF INVENTION

The present disclosure is related to a molded part that forms a retainer device. The device includes moveable members for engaging an inwardly or outwardly supported article.

### BACKGROUND

Dispensing devices for providing a fragrance to a room are known. These devices usually include a reservoir for holding a volatile liquid that contains the beneficial properties. A wick may be used to transition the volatile liquid from the reservoir to the environment. The wick is positioned with one end located in the volatile liquid and the opposite end located outside of the container. Using capillary action the liquid is drawn up the length of the wick. Once the liquid reaches the outside of the container it is vaporized and dispersed throughout the environment by simple diffusion or with the assistance of a heater, flame, fan or other device.

Numerous types of retaining devices are used to hold the wick securely in place within known dispensing devices. Known methods of securing the wick within these retaining devices include inserting a pin through the wick or using the holding device to secure the wick such that its removal is not possible. Several additional retaining methods for securing the wick have also been previously disclosed.

U.S. Pat. No. 1,198,664 to Reynolds shows a torch device having a cylindrical wick holding structure. Two barbs or points are formed in the sidewall of the holder that engage the wick and prevent movement back into the container.

U.S. Pat. No. 367,421 to Melchior shows a holding structure for a burner having three pivoting hooks positioned around the periphery of the wick to hold it in a desired position. The hooks are separately formed from the base structure and are pivotably attached to outwardly projecting tabs.

U.S. Pat. No. 4,437,832 to Amano et al. shows a lamp assembly having a wick supported by a first holder member and an auxiliary holder. A flexible engagement strip is attached at one end to a sidewall of the first holder. The strip includes a pawl on the free end. The pawl projects through a window in the holder and through slits in the auxiliary holder to engage an opening in the wick.

U.S. Pat. No. 7,303,143 to Davis et al. shows a bottle and wick holder combination. The tolerances of the internal openings of the wick holder are such that a frictional fit is created with the wick.

U.S. Pat. No. 6,619,560 to Chun shows a bottle and wick holding assembly wherein portions of the sidewalls of the wick holder flex inwardly upon engagement with a clip that forms an internal shoulder within the supporting element.

It is also known to create molded parts having a central hollow core. The mold for such parts includes an outer mold portion, comprising one or more members, and an inner mold portion that is moveable with respect to the outer mold portion. The mold cavity is defined between the two mold portions. A molten plastic material is injected into the mold cavity to form the molded part. After molding and cooling of the part within the mold cavity, one or both of the mold portions is/are moved relative to the other along a line of draw. Once sufficient clearance is created, the molded part is ejected from the mold.

### SUMMARY OF THE INVENTION

The invention concerns a dispensing device. In one example embodiment the device comprises a reservoir having a top defining a support opening. A retainer body is positioned within the support opening. The retainer body is defined by one or more sidewalls. The one or more sidewalls arde formed about an axial opening which is formed about a central axis. At least one engagement opening is formed in the sidewall. The at least one engagement opening is positioned substantially transverse to the axial opening. A wick is positioned within the axial opening and extends into the reservoir. At least one engagement member is pivotably connected to the sidewall within the at least one engagement opening. The engagement member is pivoted by contact with the top into a position wherein at least a portion of the engagement member is located within the axial opening and is in contact with the wick.

By way of example, the axial opening defines a substantially cylindrical bore within the retainer body. In a particular example the retainer body is co-axially formed about the central axis of the axial opening. By way of further example, the retainer body is substantially cylindrical. In a particular example embodiment a diameter of an outer surface of the retainer body varies along the length of the retainer body. By way of further example, the diameter of an outer surface of the retainer body tapers toward a first end.

In a specific example embodiment the engagement member comprises a projection. By way of example, the engagement member is substantially triangular, wherein a first corner of the triangle forms a pivot connection of the engagement member to the sidewall and wherein a second corner of the triangle comprises the projection. In a further example a plurality of the engagement members are provided. Each engagement member is pivotably connected to the sidewall within a separate one of the engagement openings in this example embodiment.

In a particular example embodiment the reservoir contains a volatile material. The material may be selected from the group consisting of fragrances and insect repellants by way of example.

The invention further encompasses a dispensing device, which, by way of example, comprises a reservoir having a top defining a support opening. A retainer is positioned within the support opening. The retainer comprises a coupling sleeve in this example. The coupling sleeve is defined by one or more sidewalls. The one or more sidewalls have an outer surface engaging the top within the support opening. The coupling sleeve has an axial opening formed about a central axis. At least one engagement opening is formed in the sidewall. The at least one engagement opening is positioned substantially transverse to the axial opening. A wick is positioned within the axial opening and extends into the reservoir. A wick engagement member is pivotably connected to the sidewall and positioned within the engagement opening such that at least a portion of the wick engagement member is pivoted into the axial opening and into contact with the wick.

In a particular example embodiment the axial opening defines a substantially cylindrical bore within the coupling sleeve. In a further example the coupling sleeve is co-axially formed about the central axis of the axial opening. In a specific example embodiment the coupling sleeve is substantially cylindrical. Further by way of example, the diameter of the outer surface of the coupling sleeve varies along the length of the sleeve. In another example the diameter of the outer surface of the coupling sleeve tapers toward a first end. By way of further example, the coupling sleeve further comprises a retaining ring defining a shoulder of the outer surface and the retaining ring is positioned at an end of the coupling sleeve. In this example the retaining ring engages the top when positioned within the support opening.

Also by way of example, the engagement member is substantially triangular. In this example a first corner of the triangle forms a pivot connection of the engagement member to the coupling sleeve. Also in this example a second corner of the triangle comprises the projection for contact with the wick. In a specific example embodiment a plurality of the engagement members are provided, the plurality of engagement members each being pivotably attached to the sidewall within separate ones of the engagement openings. By way of example the reservoir may contain a volatile material.

### BRIEF DESCRIPTION OF THE DRAWINGS

For the purpose of illustrating the invention, there is shown in the drawings one or more forms that are presently preferred; it being understood that the invention is not limited to the precise arrangements and instrumentalities shown.

FIG. 1 is a perspective view of an embodiment of a device as contemplated by the present disclosure, wherein the device is positioned within a support and wherein the device engages and retains an internally positioned article, herein in the form of a wick.

FIG. 2A is a top plan view of the molded device of FIG. 1 shown in a first or molded position

FIG. 2B is a cross sectional view of the device as taken along lines 2B-2B in FIG. 2A.

FIG. 3A is a top plan view of the molded device of FIG. 1 shown in a second position.

FIG. 3B is a cross sectional view of the device as taken along lines 3B-3B in FIG. 3A.

FIG. 4 is a bottom plan view of the molded device of FIG. 1 shown in the first or molded position.

FIG. 5 is a bottom plan view of the molded device of FIG. 1 shown in the second position.

FIG. 6 is an angled view of the molded device of FIG. 1 shown in a first, molded or engaged position.

FIG. 7 is a further angled view of the molded device of FIG. 1 shown in the second position.

FIG. 8 is a further angled view of the molded device of FIG. 1 shown in the first, molded, or engaged position.

FIG. 9 is a cross sectional view of the molded device as taken along lines 2B-2B shown in the second position, with a wick article positioned in the bore of the device.

FIG. 10 is a cross sectional view of the molded device as taken along lines 3B-3B shown in the first position, with a wick article positioned in the bore of the device and a partial cross sectional view of the support positioned around the device.

FIG. 11 is a cross sectional view of a first mold portion and a second mold portion indicating movement of one portion in relation to the other portion.

FIG. 12 is a cross sectional view of the first mold portion inserted within the second mold portion.

FIG. 13 is a cross sectional view of the first mold portion inserted within the second mold portion and a liquid molding compound injected there between to form the device.

FIG. 14 is a cross sectional view of a molded device with the first mold portion removed.

FIG. 15 is a cross sectional view of the molded device as removed from the second mold portion, and indicates the movement of the engagement members during the removal process.

FIG. 16 is an external side view of the molded device as removed from the mold portions.

### DETAILED DESCRIPTION

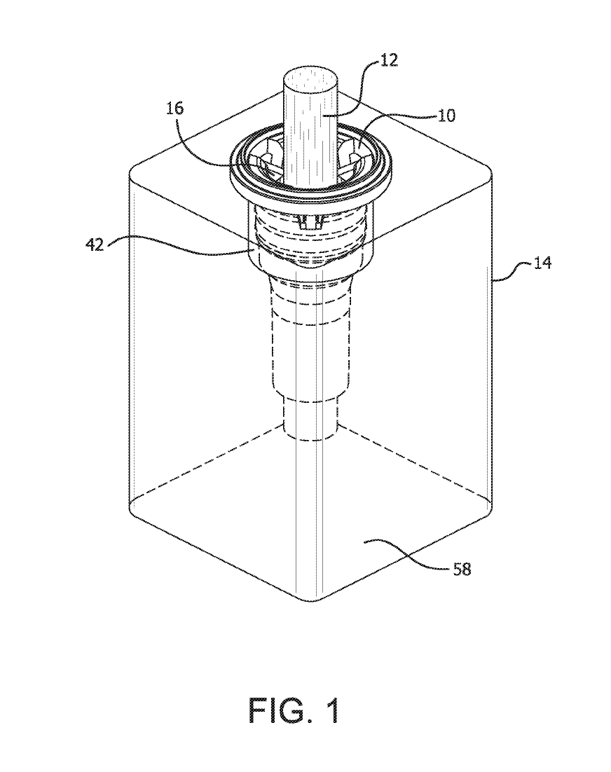

In the figures, where like numerals identify like elements, there is shown an embodiment of a molded retainer device preferably formed by injection molding. FIG. 1 shows a preferable final assembled embodiment for the molded retainer holding a wick within a support opening in a dispensing device. The molded retainer is generally designated by the numeral 10, the wick numeral 12 and the dispensing device numeral 14. When the molded retainer 10 is inserted into the support opening 42 of the dispensing device 14, engagement members 16, which are integrally formed as part of the molded retainer 10 secure the wick 12, preventing movement of the wick 12 in relation to the dispensing device 14.

Although FIG. 1 shows a preferred embodiment of the use of the molded retainer with a wick and dispensing device assembly, it is understood that the molded retainer may be useful in other fields. The method of creating a general molded retainer is described herein and generally secures a first structure in relation to a second structure.

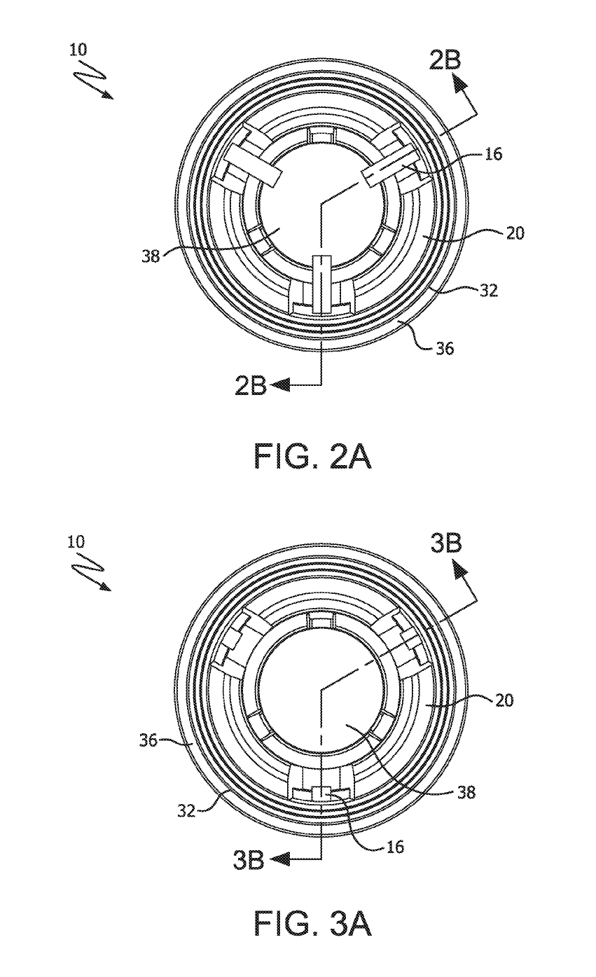

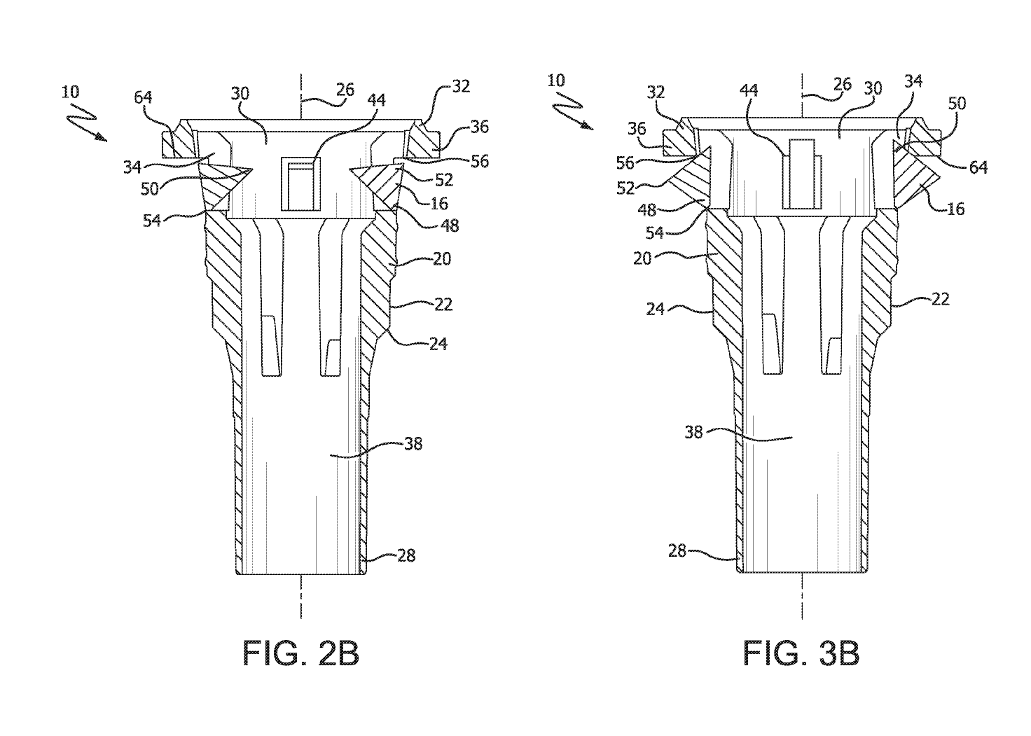

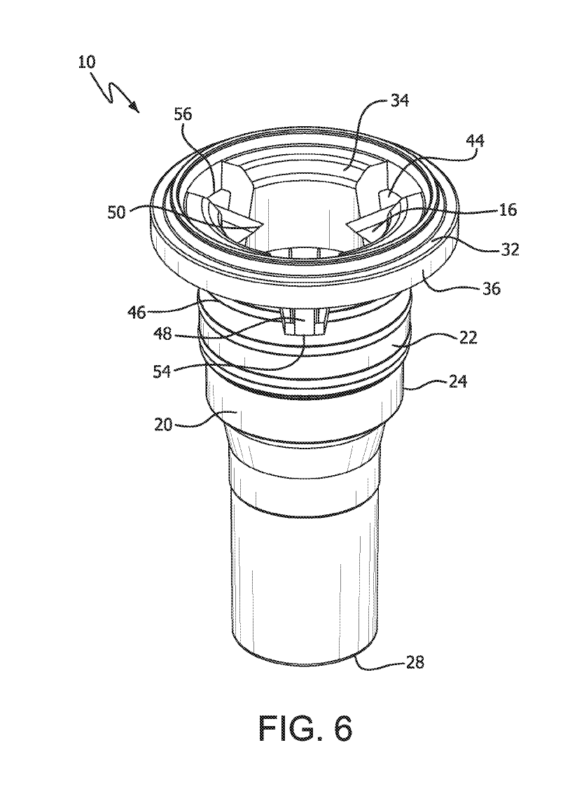

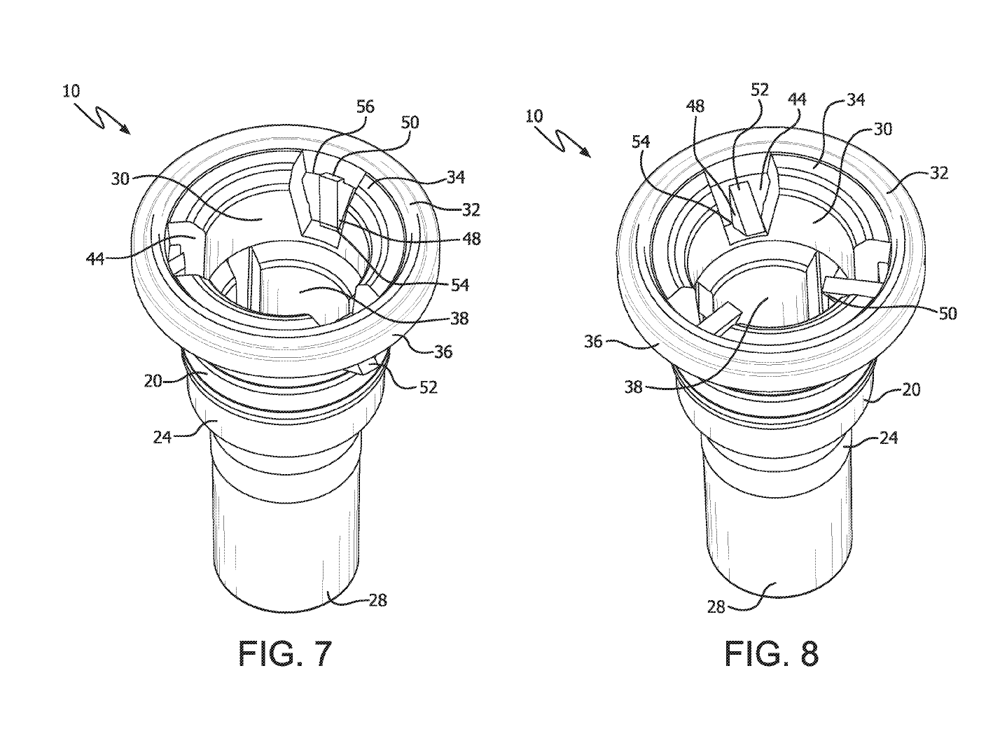

FIGS. 2-8 show a preferred embodiment of the molded retainer 10. The molded retainer 10 includes a retainer body 20 having one or more sidewalls 22. The number of sidewalls 22 may be used to create the desired shape of the retainer body, or may also be dependent on the type of molding process used to create the retainer. In the preferred embodiment shown in the figures, only one sidewall 22 is used, resulting in a substantially cylindrical retainer body 20 as shown by the top and bottom views. The diameter of the outer surface 24 of the retainer body 20 as measured along a line perpendicular to the central axis 26 of the axial opening 30 may vary along the length of the retainer body 20. Moreover, the diameter of the outer surface 24 preferably tapers towards a first end 28. This configuration allows for easy removal of a first mold portion, as well as relatively easy insertion into a dispensing device.

It is contemplated that any number of sidewalls may be used such that any shape cross section for the outer surface of the retainer body as viewed from the top and/or bottom is possible. It is further understood that the cross-sectional shape of the outer surface of the retainer body does not have to be uniform along the entire length of the molded retainer. The retainer body 20 may also have a retaining ring 32 located at the outer surface 24 of the retainer body 20 at a second end 34, defining a shoulder 36. The retaining ring 32 may engage with the top 40 of the dispensing device 14 when the molded retainer 10 is positioned within the support opening 42 of the dispensing device 14. The shape of the retaining ring 32 is preferably consistent with the shape of the retainer body 20, but consistency is not required and any shape retaining ring is understood.

The sidewall(s) 22 forms an axial opening 30 about a central axis 26. In a preferred embodiment the axial opening 30 defines a cylindrical bore 38 within the retainer body 20. The diameter of the cylindrical bore 38 remains relatively constant throughout the entire length of the retainer body 20. Although a cylinder is preferred, any shape cross section for the bore 38 as viewed from the top or bottom is contemplated. It is further understood that the cross-sectional shape of the bore does not have to be uniform for the entire length of the molded retainer 10 and also does not have to have the same cross-sectional shape as the outer surface 24 of the retainer body 20. The cylindrical bore, as well as the retainer body 20 are co-axially formed about the central axis 26 of the axial opening 30.

One or more engagement opening(s) 44 are formed in the one or more sidewall(s) 22 and are positioned substantially transverse to the axial opening 30. In a preferred embodiment, three engagement openings 44 may be located equidistant from each other around the upper portion 46 of the sidewall 22. At least one engagement member 16 is integrally formed with the retainer body 20 and pivotably connected to the sidewall 22. The engagement member 16 is formed within the engagement opening 44. A preferred embodiment may have three engagement members 16, respectively formed within three preferred engagement openings 44. The use of a single engagement opening 44 and engagement member 16 is also contemplated as well as a plurality of engagement openings and members up to the amount that is capable of being located around the circumference of the retainer body 20. More than one engagement member 16 may also be located in a single engagement opening 44. Additionally, one engagement opening 44 containing an engagement member 16 may be located at the same height along the length of the retainer body 20 as another engagement opening 44.

The engagement member 16 has a preferable triangular shape although any shape may be provided. A first portion of the member is integrally formed with the retainer body 20 at a living hinge 54 or the like and a second portion formed to engage an article within the bore 38. The preferred triangular shape may have a first corner 48, a second corner 50 and a third corner 52. The first corner 48 preferably forms a connection with the retainer body 20. The second corner 50 preferably forms the projection to secure an article within the bore 38 of the molded retainer 10. The connection between the first corner 48 and the retainer body 20 forms the hinge 54 and is integrally molded with the retainer body 20. The hinge 54 is configured to allow the engagement member 16 to pivot between a first position and a second position, with the first position corresponding to a molded and/or engaged position.

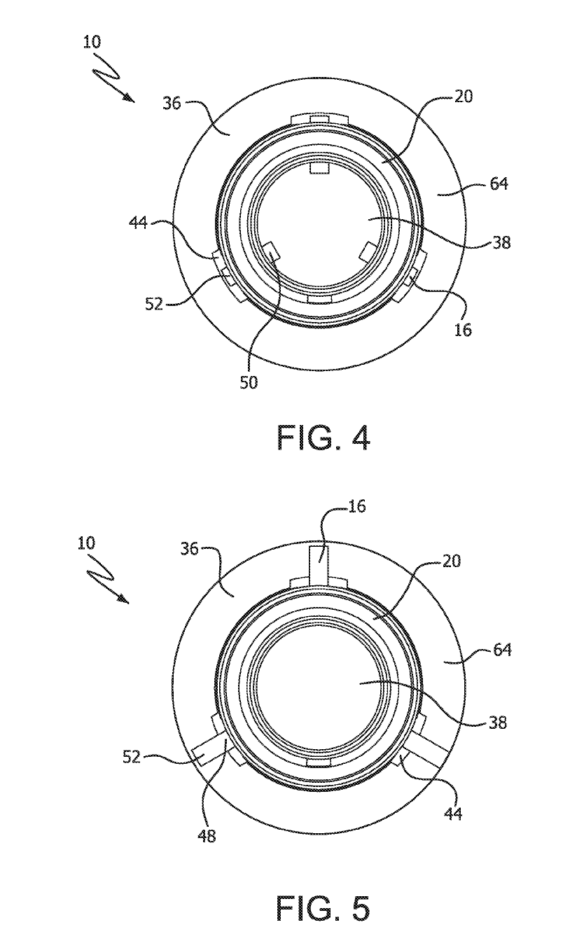

Specifically, FIGS. 2, 4 and 6 show the molded retainer 10 with the engagement members 16 in a preferred first, molded position. In the molded position, as formed, the engagement members 16 have a portion located inside of the axial opening 30. As shown, the inside portion is the second corner 50 of the engagement member 16. The second corner 50 may extend a distance into the bore 38 as depicted in FIG. 4. This distance should be sufficient to at least contact an article inserted within the bore 38.

FIGS. 3, 5 and 7 show the molded retainer 10 with the engagement members 16 in a preferred second position. After the molding process is complete and the molded retainer 10 is released, the engagement members 16 will be in the second position. The releasing of the molded retainer 10 from the second mold portion causes the engagement member(s) 16 to move from the first position to the second position. The first corner 48 remains substantially in one location and only moves rotationally about the living hinge 54. The pivoting movement about the first corner 48 may cause the second corner 50 to no longer be located within the axial opening 30. In the second position, a line extending between the second corner 50 and the first corner 48 may be substantially vertical and a majority of the engagement member 16 may be located outside of the axial opening 30. No part of the molded retainer 10 should be located within the bore 38 when the engagement members 16 are in the second position.

In the second position the second corner 50 may prevent further movement of the engagement member 16 towards the outside of the retainer body 20. The dimension of the engagement member 16 and the engagement opening 44 may cause the second corner 50 to contact the retaining ring 32 at the top 56 of the engagement opening 44 when in the second position. In the second position, the majority of the weight of the engagement member 16 may be located outside of the retainer body 20. Therefore, the contact between the second corner 50 and the retaining ring 32 may stop any further rotation about the living hinge 54 that may be caused by gravity or other force. Movement of the engagement member 16 past a certain point could inhibit the ability of the user to insert the molded retainer 10 into a support structure. A full range of rotation about the hinge 54 is contemplated. The second corner 50 does not need to provide a stoppage function. In which case the engagement member 16 may fully rotate about the living hinge 54 and the third corner 52 of the engagement member 16 may contact the outer surface 24 of the retainer body 20 in the second position.

FIG. 8 shows a preferred embodiment of the molded retainer with the engagement members 16 in a first engaged position. One preferable configuration for the molded retainer 10 has the engagement members 16 molded in a slightly open position, as included in FIGS. 2, 4, and 6. This position may allow for easier removal of the molded retainer 10 during the molding process. When the engagement members 16 are forced to engage an article within the bore 38, they may be fully closed. Specifically, the second corner 50 may be allowed to point at an angle slightly below horizontal, which may provide a more secure connection between the molded retainer 20 and an article within the bore 38. However, the position of the engagement member in a molded first position may also be the same as the position of the engagement member in an engaged first position.

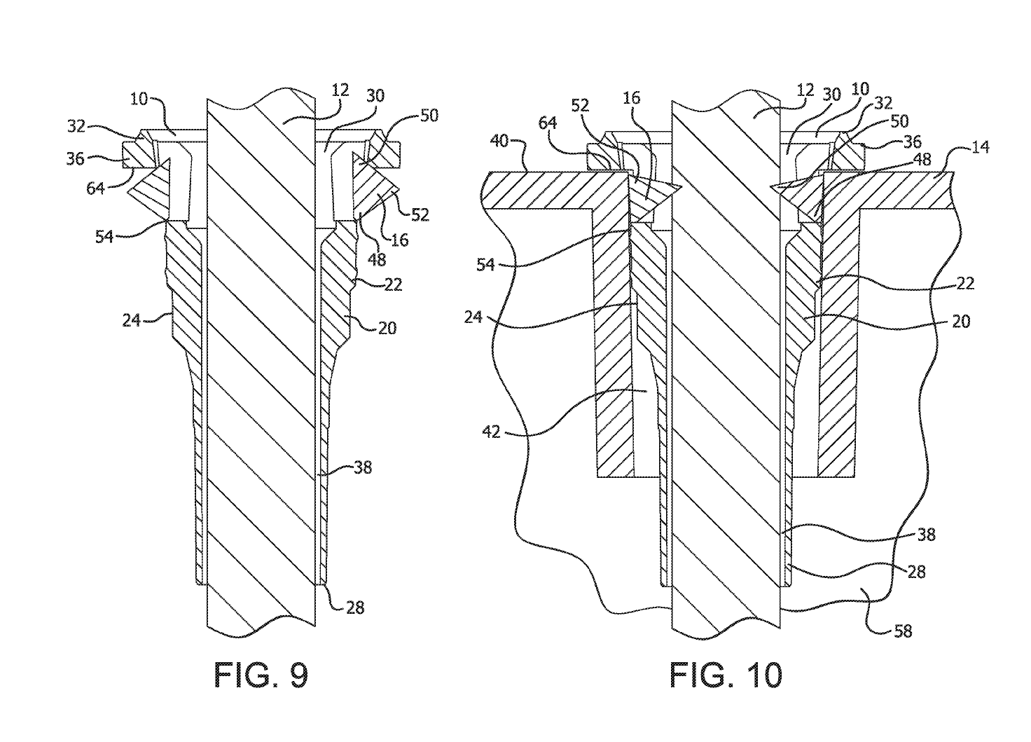

FIGS. 9 and 10 show a cross sectional view of the molded retainer 10 as it is used in a preferred embodiment. A specific use contemplated for the molded retainer 10 is to centrally support a wick 12 within a support opening 42 of a dispensing device or dispenser 14 as shown in FIG. 1. The dispenser device 14 includes a reservoir 58 for holding a volatile material, such as a fragrance or insect repellant. The wick 12 as supported by the molded retainer 10 may communicate with the contents within the reservoir 58. The contents of the reservoir 58 travel up the wick 12 to be dispersed into the environment outside of the dispenser. For such intended use the retainer body 20 may be a coupling sleeve 20 molded from a plastic material. The dimensions of the outer surface 24 of the coupling sleeve 20 are made so that an engagement with the support opening 42 of the dispenser device 14 is formed.

The molded retainer 10 is provided in the position shown in FIG. 9 after the molding process is complete. The user may then insert a wick 12 into the cylindrical bore 38, such that a sufficient amount of the wick 12 extends from the bottom 60 of the retainer 10 for communication with the contents of the reservoir 58. The wick 12 substantially fills the cylindrical bore 38 and may or may not touch the sides 62 along its entire length. With the engagement members 16 in the second position, the wick 12 is free to move in either direction along the central axis 26. The wick engagement members 16 preferably are not in contact with the wick 12.

Once the wick 12 is inserted, the retainer 10 is inserted into the support opening 42 of the dispenser 14, as shown in FIG. 10. The bottom 60 of the retainer 10 is inserted first. As the top 40 of the dispensing device 14 moves up the length of the retainer 10 it will come into contact with the engagement members 16 in the second position. Upon continued movement into the support opening 42, the top 40 of the dispensing device 14 forces the wick engagement members 16 to rotate about the hinge 54 connection. The rotation of the engagement members 16 causes the second corner 50 of each member 16 to rotate into contact with the wick 12. With continued insertion of the molded retainer 10 into the support opening 42, the second corner 50 will be pushed with increasing force into the side of the wick 12, securing the wick 12 such that it cannot be inserted further into or removed from the coupling sleeve 20. The coupling of the molded retainer 10 with the dispensing device 14 may be complete when the bottom surface 64 of the shoulder 36 of the retaining ring 32 contacts the top 40 of the dispensing device 14.

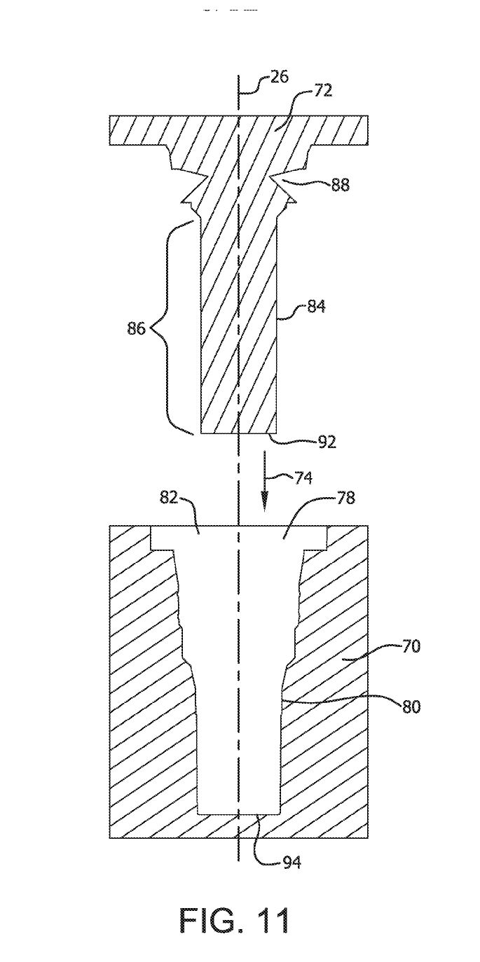

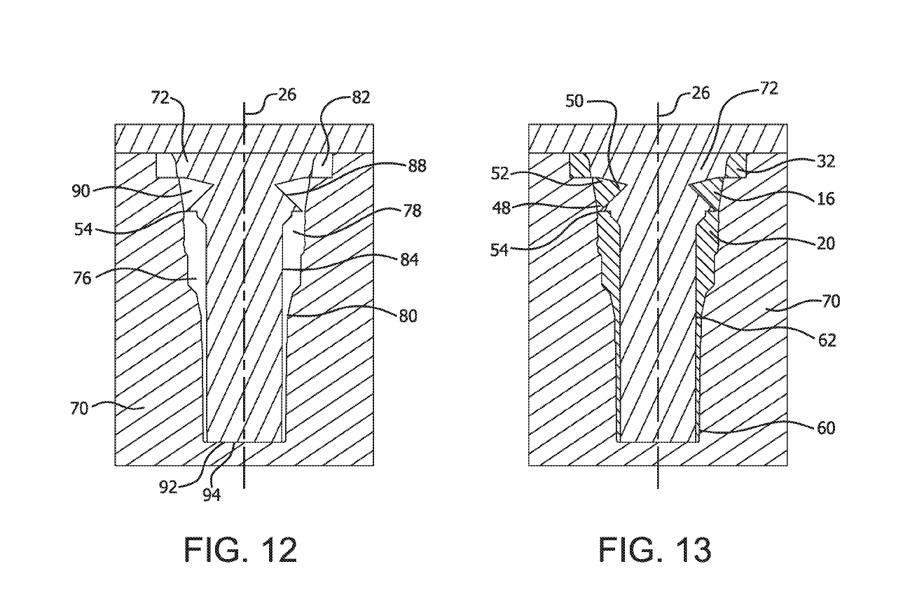

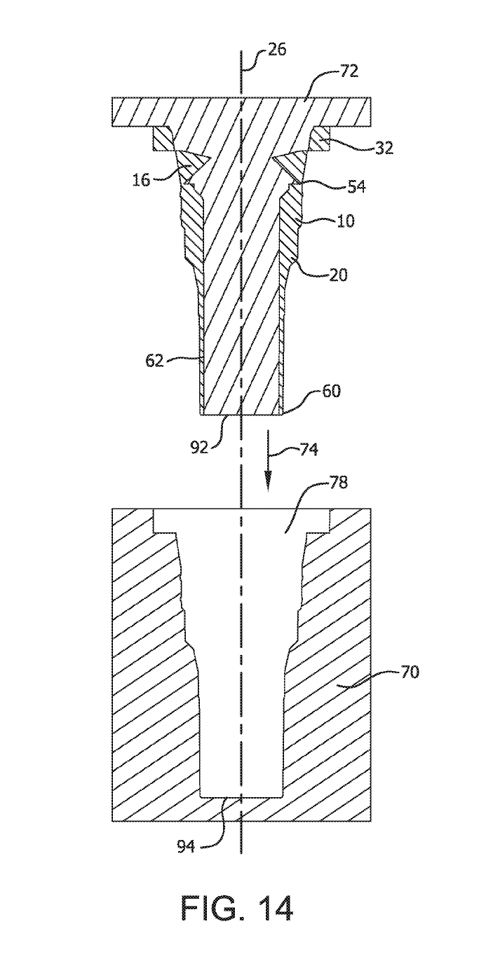

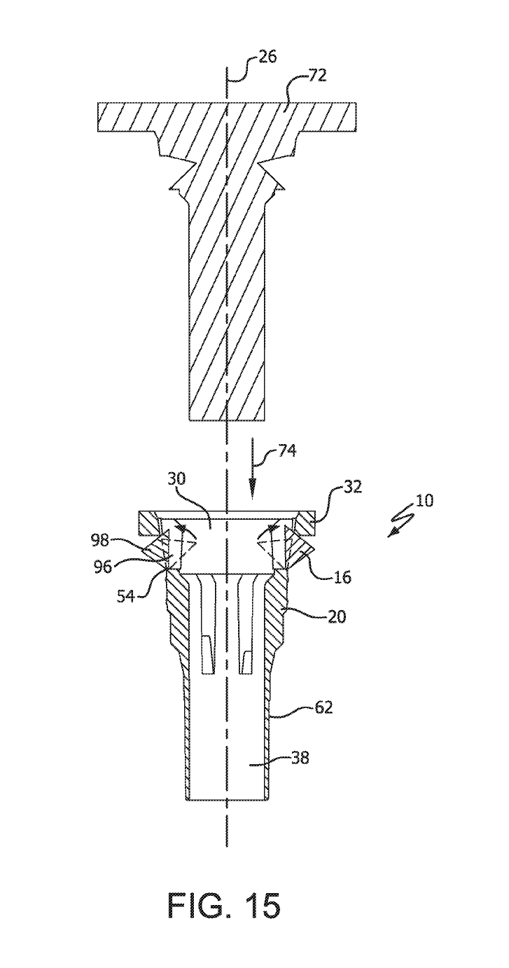

FIGS. 11-15 show the molding process used to create a molded retainer having pivotably attached engagement members 16. Preferably the molded retainer is formed by an injection molding process using a molten plastic. As shown by FIG. 11 two mold sections are provided to create the entire molded retainer. Namely, a first outer mold portion 70 and a second inner mold portion 72 are provided and at least one mold portion is movable with respect to the other mold portion along a line of draw 74. The mold portions 70 and 72 may be positioned with respect to each other defining a cavity 76 between the mold portions. The cavity 76 defines the structure of the molded retainer.

The outer mold portion 70 is provided having a mold opening 78. The inner surface 80 of the mold opening creates the outer periphery of the molded retainer. The specific surface contour of the inner surface 80 of the outer mold opening 70 creates the shape of the sidewalls of the molded retainer. Specifically, the surface contour of the outer mold opening 70 is shaped such that the outer mold portion 70 is removable from the formed product along a line of draw 74, which extends along the central axis 26.

In a preferred embodiment, the mold opening 78 is cylindrical in shape, resulting in a cylindrically shaped molded retainer. The mold opening 78 may also have a cross-sectional diameter as measured along a line perpendicular to the central axis 26 that varies with the depth from the top of the opening 82. Preferably, the diameter of the mold opening 78 decreases as the depth from the top 82 of the mold opening 78 increases. The decreasing diameter for the mold opening 78 permits the outer mold portion 70 to be removable from the molded retainer along the line of draw 74.

A second inner mold portion 72 is also provided. The inner mold portion 72 is shaped to act as the male coupling piece and fit within the mold opening 78 of the outer mold portion 70 to define the cavity 76 there between. The mold cavity 76 defines the retainer body. The retainer body being defined by one or more sidewalls. The mold cavity 76 also defines the engagement openings located in the sidewall of the molded retainer. The inside surface of the sidewalls is created from the contour of the outer surface 84 of the inner mold portion 72. The inner mold portion 72 defines an axial opening within the molded retainer, as well as a bore located substantially about the central axis 26 of the molded retainer. The inner mold portion 72 is preferably cylindrical in shape, which results in creating a preferable cylindrically-shaped bore within the molded retainer. The diameter of the inner mold may remain substantially constant along its lower portion 86, such that the diameter of the lower portion of the bore within the molded retainer is substantially constant.

Indentations 88 within the outer surface 84 of the inner mold 72 create the openings 90 in the mold cavity 76 that define at least one engagement member 16 located in a first position. The openings 90 may be made such that the engagement member 16 is integrally formed with the retainer body 20 and pivotably connected about a living hinge 54 within an engagement opening 44 located within the retainer body 20. The openings 90 are preferably triangular in shape, with a first corner 48 of the triangle being the location of the living hinge 54 connection. With the engagement member in the first position, as molded, the second corner 50 extends within the axial opening, which during the molding process is located within the inner mold portion 72 and is defined by the indentation 88. The location of the engagement member 16 in this position prevents the inner mold portion 72 from being removed along the line of draw 74 while the outer mold portion 70 is still in position around the inner mold portion 72.

For creating the molded product, the inner mold portion 72 is inserted within the mold opening 78 in the outer mold portion 70 along the line of draw 74. The bottom of the inner mold portion 92 contacts the bottom of the mold opening 94 in the outer mold portion 70, such that the bore within the molded retainer will be open at both the top and the bottom. Once the mold portions 70 and 72 are aligned with each other, a molten plastic or other liquid molding material is injected into the cavity 76. A standard injection molding process along with any known molding material may be used to create the molded retainer 10, including both metals and plastics. The molten material, preferably plastic, is allowed to cool and solidify prior to moving either mold portion 70 or 72.

As discussed above, the mold cavity 76 creates the engagement member 16 in the first position as integrally formed with the retainer body 20. The molded first position 96 of the engagement member 16 is preferably slightly opened, such that in use, the engagement member 16 may close more fully creating a more secure hold. The engagement member 16 is located within the inner mold portion 72 such that the inner mold portion 72 cannot be removed immediately after the molding process is complete and the molded retainer 10 is solidified. To remove the molded retainer 10 from the mold portions 70 and 72 a two step process is used.

As an initial step, the movable mold portion, which in this embodiment included in the figures is the outer mold portion 70, is removed along the line of draw 74. The outer mold portion 70 does not have any restrictions to its movement along this line. Once the movable mold portion is removed, the molded retainer 10 is ejected from the other mold portion 72 along the same line of draw 74. As the molded retainer 10 is ejected, the engagement member 16 is pivoted from the first position 96 to a second position 98. The second position 98 is located within the line of draw 74 of the moveable mold portion.

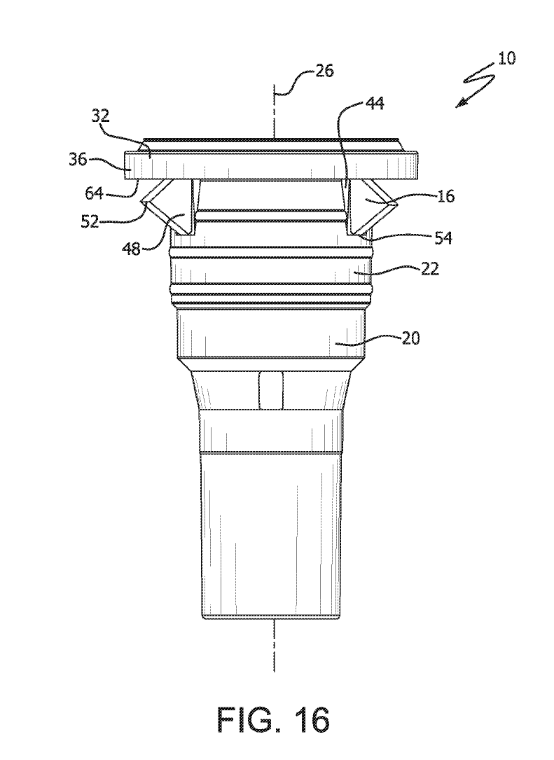

FIG. 16 shows an embodiment of the complete molded retainer 10 after removal from the inner 72 and outer 70 mold portions. The molded retainer 10 comprises a retainer body 20 formed by at least one sidewall 22. The retainer body 20 may have a generally cylindrical shape. The sidewalls may comprise engagement openings 44, with engagement members 16 therein. The sidewalls 22 may also be formed about an axial opening. The axial opening may define a cylindrical bore that extends the entire length of the molded retainer 10 and may be formed about a central axis 26. After molding is complete, the molded retainer 10 is in an open configuration, with the engagement members 16 in the second position with at least a portion of the engagement member 16 located outside of the axial opening.

Although the preferred embodiment described herein shows the molding process forming the molded retainer having engagement members in the first position. It is also contemplated that the engagement members may be molded in the second position and moved to the first position by the ejection step. In a preferable configuration for this positioning, the pivot point of the engagement member is moved from the bottom outside corner to the top outside corner of the triangle. The inner mold may be removed first, followed by ejection of the molded retainer from the outer mold portion. When the molded retainer is ejected along the line of draw, the engagement members are moved from the second position with a portion located on the outside of the axial opening to the first position with a portion located on the inside of the axial opening. In this embodiment, the retainer secures structures on its outside surface.

The present disclosure shows and describes one or more exemplary embodiments. It should be understood by those skilled in the art from the foregoing that various other changes, omissions and additions may be made therein, without departing from the spirit and scope of the contemplated invention, with the scope of the invention being defined by the foregoing claims. Further, the terms herein are used in a generic and descriptive sense and are not necessarily for purposes of limitation. The scope of the invention is set forth in the following claims.

What is claimed is:

1. A dispensing device, comprising:
a reservoir having a top defining a support opening;
a retainer body positioned within said support opening, said retainer body defined by one or more sidewalls, said one or more sidewalls formed about an axial opening formed about a central axis, and at least one engagement opening formed in said sidewall, said at least one engagement opening positioned substantially transverse to said axial opening;
a wick positioned within said axial opening and extending into said reservoir; and
at least one engagement member pivotably connected to said sidewall within said at least one engagement opening, said engagement member pivoted by contact with said top into a position wherein at least a portion of said engagement member is located within said axial opening and is in contact with said wick.

2. The dispensing device according to claim 1, wherein said axial opening defines a substantially cylindrical bore within said retainer body.

3. The dispensing device according to claim 2, wherein said retainer body is co-axially formed about said central axis of said axial opening.

4. The dispensing device according to claim 3, wherein said retainer body is substantially cylindrical.

5. The dispensing device according to claim 4, wherein a diameter of an outer surface of said retainer body varies along said length of said retainer body.

6. The dispensing device according to claim 5, wherein said diameter of an outer surface of said retainer body tapers toward a first end.

7. The dispensing device according to claim 1 wherein said engagement member comprises a projection.

8. The dispensing device according to claim 7 wherein said engagement member is substantially triangular, wherein a first corner of said triangle forms a pivot connection of said engagement member to said sidewall and wherein a second corner of said triangle comprises said projection.

9. The dispensing device according to claim 1 wherein a plurality of said engagement members are provided, each said engagement member being pivotably connected to said sidewall within a separate one of said engagement openings.

10. The dispensing device according to claim 1, wherein said reservoir contains a volatile material selected from the group consisting of fragrances and insect repellants.

11. A dispensing device, comprising:
a reservoir having a top defining a support opening;
a retainer positioned within said support opening, said retainer comprising a coupling sleeve, said coupling sleeve defined by one or more sidewalls, said one or more sidewalls having an outer surface engaging said top within said support opening, said coupling sleeve having an axial opening formed about a central axis, and at least one engagement opening formed in said sidewall, said at least one engagement opening positioned substantially transverse to said axial opening;
a wick positioned within said axial opening and extending into said reservoir;
a wick engagement member pivotably connected to said sidewall and positioned within said engagement opening such that at least a portion of said wick engagement member is pivoted into said axial opening and into contact with said wick.

12. The dispensing device according to claim 11 wherein said axial opening defines a substantially cylindrical bore within said coupling sleeve.

13. The dispensing device according to claim 12 wherein said coupling sleeve is co-axially formed about said central axis of said axial opening.

14. The dispensing device according to claim 13 wherein said coupling sleeve is substantially cylindrical.

15. The dispensing device according to claim 14 wherein a diameter of said outer surface of said coupling sleeve varies along said length of said sleeve.

16. The dispensing device according to claim 15 wherein said diameter of said outer surface of said coupling sleeve tapers toward a first end.

17. The dispensing device according to claim 11 wherein said coupling sleeve further comprises a retaining ring defining a shoulder of said outer surface, said retaining ring positioned at an end of said coupling sleeve, wherein said retaining ring engages said top when positioned within said support opening.

18. The dispensing device according to claim 11 wherein said engagement member is substantially triangular, wherein a first corner of said triangle forms a pivot connection of said engagement member to said coupling sleeve and wherein a second corner of said triangle comprises said projection for contact with said wick.

19. The dispensing device according to claim 11 wherein a plurality of said engagement members are provided, said plurality of engagement members each being pivotably attached to said sidewall within separate ones of said engagement openings.

20. The dispensing device according to claim 11, wherein said reservoir contains a volatile material.
